(19) United States
(12) Patent Application Publication
(10) Pub. No.: US 2016/0291001 A1
(43) Pub. Date: Oct. 6, 2016

(54) CONDUCTIVE HYDROGELS FOR AFFINITY SENSING

(71) Applicant: THE REGENTS OF THE UNIVERSITY OF CALIFORNIA, OAKLAND, CA (US)

(72) Inventors: ALEXANDER REVZIN, DAVIS, CA (US); ZIMPLE MATHARU, DAVIS, CA (US); JUNGMOK YOU, DAVIS, CA (US); LYDIA KWON, DAVIS, CA (US); YING LIU, DAVIS, CA (US); DONG-SIK SHIN, DAVIS, CA (US)

(73) Assignee: THE REGENTS OF THE UNIVERSITY OF CALIFORNIA, OAKLAND, CA (US)

(21) Appl. No.: 15/186,089

(22) Filed: Jun. 17, 2016

Related U.S. Application Data
(63) Continuation of application No. PCT/US2014/071596, filed on Dec. 19, 2014.
(60) Provisional application No. 61/918,099, filed on Dec. 19, 2013.

(51) Int. Cl. G01N 33/543; G01N 27/327; C08L 65/00; G01N 33/545; G01N 33/68

(52) U.S. Cl. CPC G01N 33/5438; G01N 33/545; G01N 33/54353; G01N 33/6866; C08L 65/00; G01N 27/3278; G01N 27/3277; G01N 2333/35; G01N 2333/57; G01N 2800/26

(57) ABSTRACT

The present invention provides a sensor for detection of disease markers, and methods for detecting a disease marker, using a conductive hydrogel modified with a biorecognition element.

### CROSS-REFERENCES TO RELATED APPLICATIONS

The present application is a continuation of PCT/US2014/071596, filed Dec. 19, 2014, which application claims the benefit of priority to U.S. Provisional Patent Application No. 61/918,099 filed Dec. 19, 2013, all of which applications are incorporated herein by reference in their entireties.

### STATEMENT AS TO RIGHTS TO INVENTIONS MADE UNDER FEDERALLY SPONSORED RESEARCH AND DEVELOPMENT

This invention was made with Government support under Grant No. EFRI-0937997 awarded by the National Science Foundation. The Government has certain rights in this invention.

### BACKGROUND OF THE INVENTION

Conducting polymers hold significant promise in biomolecular electronics as electrode coatings and provide high electrical conductivity. However, they are characterized by inherently poor mechanical properties. Incorporation of hydrogels with conducting polymers may act to modulate and improve mechanical properties, as well as provide a non-fouling surface and a depot for water soluble, bioactive agents. Besides this, blends of electro-active polymer and biocompatible hydrogel provide three-dimensional constructs for greater biomolecule immobilization with enhanced sensitivity and hence are promising materials for biosensor development.

Among all known conducting polymers, poly(3,4-ethylenedioxythiophene) (PEDOT) exhibits very low intrinsic cytotoxicity and display no inflammatory response upon implantation, making them ideal for biosensing and bioengineering applications. Polyethylene glycol (PEG) is a biocompatible polymer known for its excellent non-fouling properties. PEG has been used widely to minimize unwanted protein adsorption and cell attachment in tissue engineering and biosensor development. We believe that the amalgam of properties of PEDOT and PEG may provide promising biosensing materials.

### BRIEF SUMMARY OF THE INVENTION

In one embodiment, the present invention provides a sensor having a substrate; at least one electrode contacting the substrate; a nanoporous membrane covering the electrode; and a biorecognition element selected from the group consisting of a peptide, an antibody, an enzyme, and an aptamer, wherein the biorecognition element is covalently bound to the electrode, or covalently bound to a PEDOT random copolymer embedded within the nanoporous membrane, the PEDOT random copolymer having a structure according to Formula I:

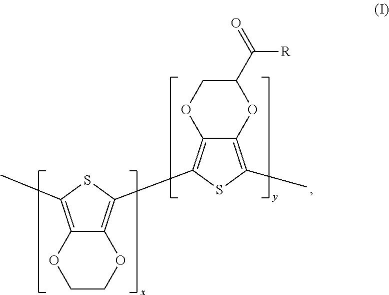

wherein each R is independently selected from the group consisting of —OH and the biorecognition element, wherein at least one R is the biorecognition element, and x and y are independently an integer of from about 1 to about 1000, wherein the sum of x and y is an integer of from about 2 to about 1000.

In another embodiment, the present invention provides a method for detecting a disease marker in a biological sample comprising contacting a sensor of the present invention with the biological sample and detecting the binding of the disease marker to a biorecognition element, thereby detecting the disease marker.

In another embodiment, the present invention provides a conductive hydrogel comprising a covalently cross-linked poly(ethylene glycol) (PEG) hydrogel; and a poly(3,4-ethylenedioxythiophene) (PEDOT) random copolymer embedded within the PEG hydrogel, the PEDOT random copolymer having a structure according to Formula I:

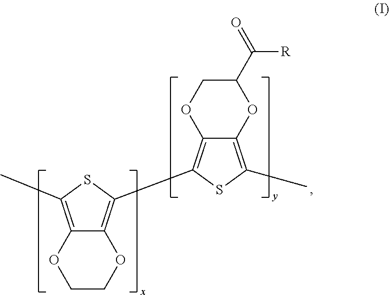

wherein each R is independently selected from the group consisting of —OH and a biorecognition element selected from the group consisting of a peptide, an antibody, an enzyme, and an aptamer, wherein at least one R is the biorecognition element, and x and y are independently an integer of from about 1 to about 1000, wherein the sum of x and y is an integer of from about 2 to about 1000.

In another embodiment, the present invention provides a method for preparing the conductive polymer of the present invention, comprising contacting a PEG-diacrylate and a photoinitiator under polymerization conditions suitable to form a PEG hydrogel; contacting the PEG hydrogel with a solution comprising 3,4-ethylenedioxythiophene (EDOT) and 2,3-dihydrothieno[3,4-b][1,4]dioxine-2-carboxylic acid (EDOT-COOH) under electropolymerization conditions sufficient to form a poly(3,4-ethylenedioxythiophene) (PEDOT) random copolymer of Formula I embedded within the PEG hydrogel:

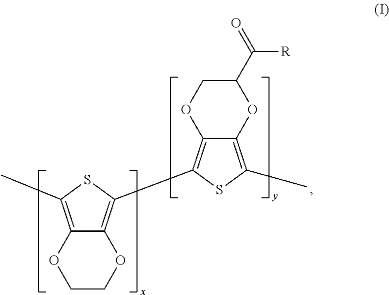

wherein each R is —OH, and x and y are each an integer of from about 1 to about 1000, wherein the sum of x and y is an integer of from about 2 to about 1000; and contacting the hydrogel with a biorecognition element under conditions sufficient to covalently bind the biorecognition element to the PEDOT random copolymer, thereby forming the PEDOT random copolymer of Formula I wherein at least one R is a biorecognition element selected from the group consisting of a peptide, an antibody, and an aptamer, thereby preparing the conductive hydrogel of the present invention.

### BRIEF DESCRIPTION OF THE DRAWINGS

FIG. 1 shows fabrication of patterned electrodes and polymerization of porous PEG hydrogel.

FIG. 2A, FIG. 2B, and FIG. 2C show polymerization of EDOT/EDOT-COOH into PEG hydrogel.

FIG. 3A and FIG. 3B show cyclic voltammograms (scan rate 50 mV/s, 1×PBS) of PEG hydrogel electrodes without (3A) and with (3B) PEDOT incorporation.

FIG. 4A and FIG. 4B show the response of antibody immobilized PEDOT-PEG hydrogel towards different recombinant concentrations. FIG. 4A shows a plot showing suppression in reduction peak in the cyclic voltammogram of PEDOT-PEG hydrogel with increasing recombinant B-IFN-γ concentrations. FIG. 4B shows values of reduction current as a function of recombinant B-IFN-γ concentrations.

FIG. 5 shows a multi-well plate array with on-plate electrode design and assembly. (1) Bottom layer—patterned electrodes. These include, working counter and reference. 2) 2nd layer (blue), nanoporous membrane preventing cells from physiological liquids from adsorbing onto electrodes. 3) Microwells laser cut in plastic and glued onto the previous substrate and 4) plastic seal to prevent evaporation of physiological liquid being tested.

FIG. 6A, FIG. 6B, and FIG. 6C show an eight well plate electrode array fabrication with the contact pads and connection leads (FIG. 6A) and screen printed reference electrode with Ag/AgCl paste, the red circle indicates each well plate including the working, counter and reference electrode (FIG. 6B). Assembly of nanoporous membrane and PDMS microwell membrane (FIG. 6C).

FIG. 7A shows the fabrication of PEDOT/PEG conductive hydrogel via photo-polymerization of PEG and electro-polymerization of PEDOT inside prepared PEG.

FIG. 7B shows the binding of bovine IFN-γ to antibody immobilized conductive hydrogel results in electrochemical signal decrease.

FIG. 8A, FIG. 8A, and FIG. 8C show features of electrodes. FIG. 8A shows a patterned Au electrode array and electrodes before (FIG. 8B) and after (FIG. 8C) electropolymerization of EDOT/PEG conductive hydrogel via photo-polymerization of PEG and electro-polymerization of PEDOT inside prepared PEG on ITO electrodes (scale bar: 500 μm).

FIG. 9A, FIG. 9B, FIG. 9C, and FIG. 9D show SEM images revealing the surface morphology of Au electrode (FIG. 9A), PEG (FIG. 9B), PEDOT (FIG. 9C) and PEDOT/PEG (FIG. 9D) conductive hydrogel.

FIG. 10A, FIG. 10B, FIG. 10C, and FIG. 10D show characterization of conductive hydrogel: (FIG. 10A) Cyclic voltammograms (CV) showing comparison of only PEG and PEDOT/PEG surface. CV of PEDOT/PEG matrix shows intrinsic oxidation and reduction of PEDOT with 100 fold enhanced current values as compared to only PEG matrix. (FIG. 10B) CV of PEDOT and PEDOT/PEG electropolymerized on Au surface. (FIG. 10C) The B-INF-γ antibody immobilized conducting polymer hydrogel surface was challenged with different concentrations of recombinant bovine IFN-γ and the cyclic voltammograms were recorded. Capture of IFN-γ causes the decrease in Iₚ values. (FIG. 10D) A calibration curve was obtained based on the concentration of B-INF-γ versus Iₚ values.

FIG. 11A, FIG. 11B, FIG. 11C, FIG. 11D, and FIG. 11E show performance test of the conductive hydrogel sensor: (FIG. 11A) Electrochemical current peaks did not change when the PEDOT/PEG is challenged with whole blood, (FIG. 11B) Nonspecific cytokines or (FIG. 11C) human INF-γ did show significant electrochemical signals with the sensor. (FIG. 11D) Electrochemical signal reduction responded to bovine IFN-γ in plasma samples. Correlation between ELISA results and electrochemical signal reduction. (FIG. 11E) Real time detection of cell-released bovine IFN-γ from bovine PBMCs.

FIG. 12A, FIG. 12B, and FIG. 12C show AFM mechanics for PEDOT (FIG. 12A), PEG (FIG. 12B) and PEDOT+PEG (FIG. 12C).

FIG. 13A, FIG. 13B, FIG. 13C, and FIG. 13D show optimization of PEDOT deposition as a function of charge applied.

FIG. 14A and FIG. 14B show quantitation of carboxylic groups on conductive hydrogel via Toluidine Blue staining assay.

FIG. 15A and FIG. 15B show fluorescence images on ITO electrodes at various concentrations of bovine INF-γ.

FIG. 16A and FIG. 16B show INF-γ detection spiked with bovine blood/PBS (1:1).

FIG. 17 shows a schematic drawing of the sensor of the present invention, including the location of the reference, working and counter electrodes.

FIG. 18A and FIG. 18B show various embodiments of the biosensor (100) of the present invention in cross-section, including a substrate (110), electrodes (120), a nanoporous membrane (130) with biorecognition elements (140), and a self-assembled monolayer (150) on the substrate.

FIG. 19A and FIG. 19B shows various embodiments of the biosensor (200) of the present invention in cross-section, including a substrate (210), electrodes (220), a nanoporous membrane (230), with biorecognition elements (240) linked to the electrodes via a self-assembled monolayer (250) on the electrodes.

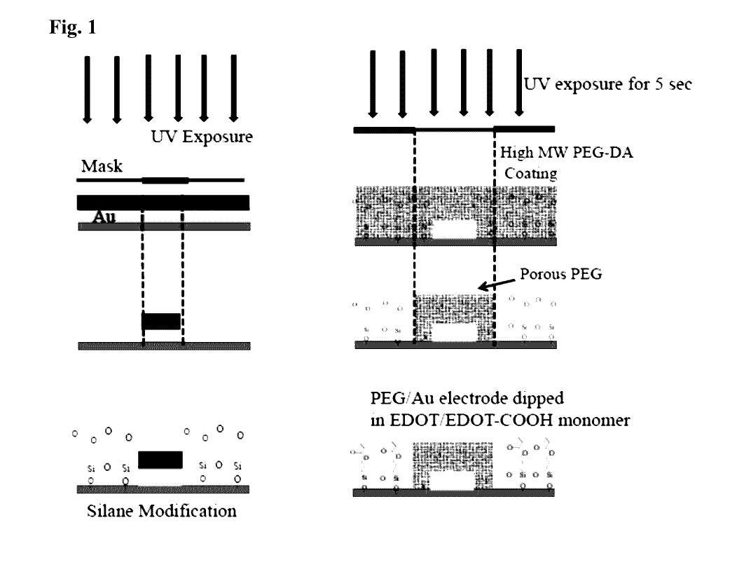

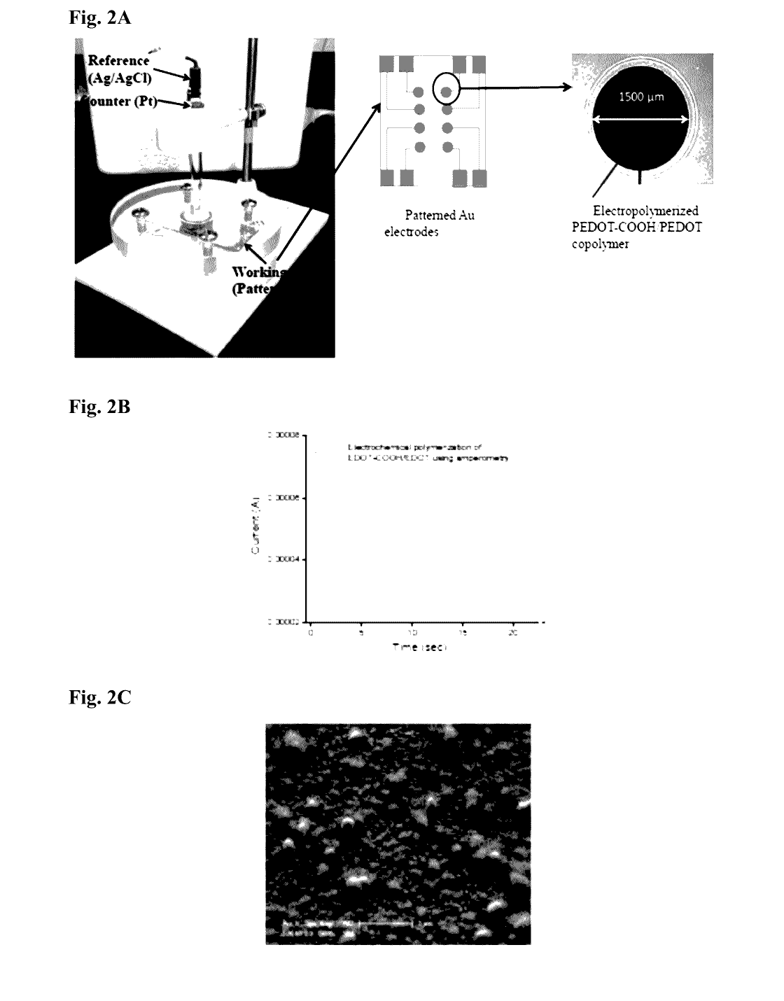

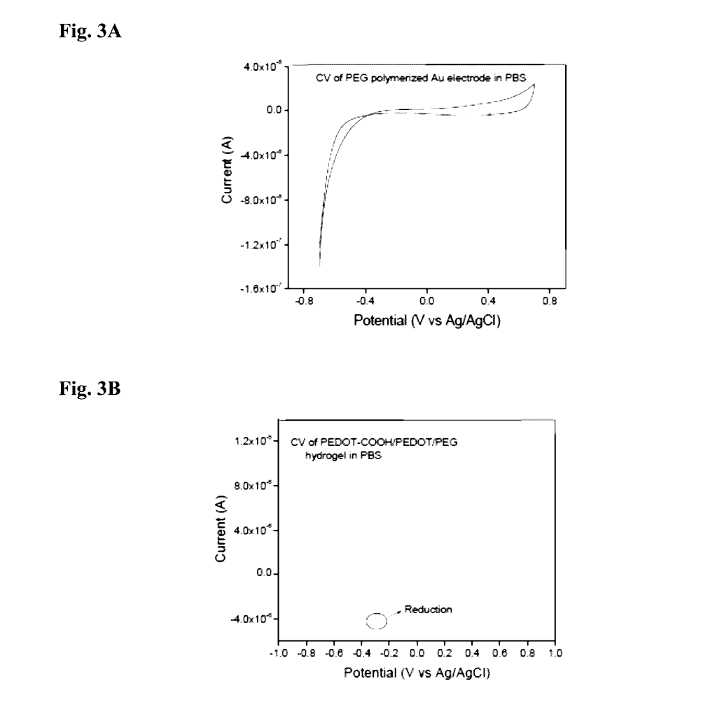

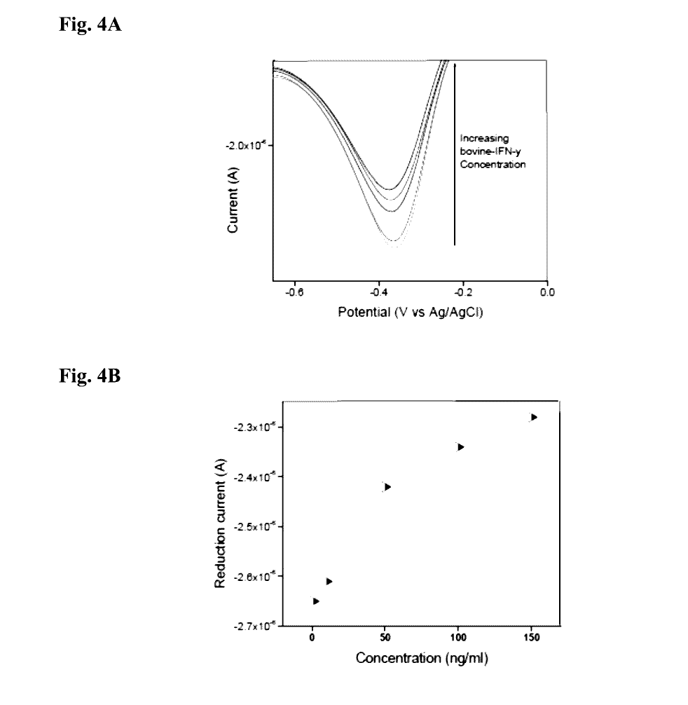

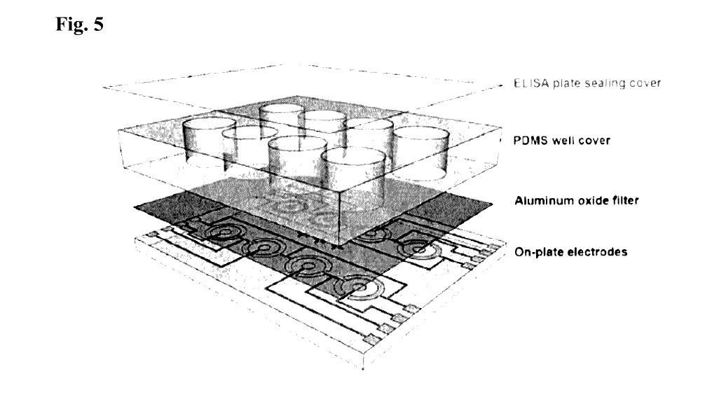

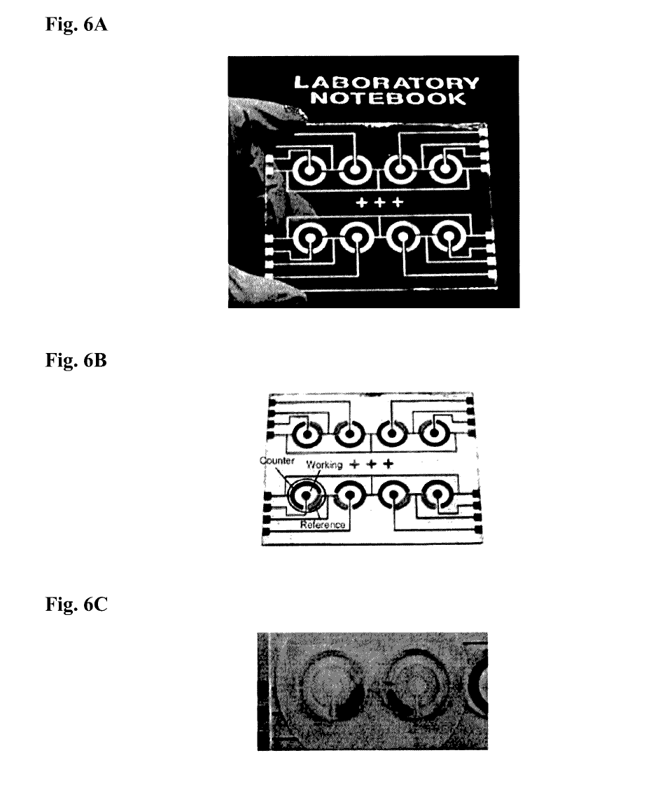

### DETAILED DESCRIPTION OF THE INVENTION

### I. General

The present invention provides a sensor for detection of biological agents and detecting of disease. The sensor includes a biorecognition element attached to a conducting polymer that covers an electrode, so that binding of the biorecognition element to the biological agent results in a change in the measured potential for the electrode, thus detecting the binding event. The sensor enables detection without the use of a label. Moreover, the conducting polymer, a PEDOT-PEG hydrogel, has improved mechanical properties and better resists delamination from the electrode due to covalent binding to the substrate surface.

### II. Definitions

“Substrate” refers to any solid material suitable for supporting the sensor of the present invention. Suitable substrates can include materials such as silicon, silicon dioxide and indium-tin-oxide. Examples of suitable substrates include, but are not limited to, glass (including controlled-pore glass), polymers (e.g., polystyrene, polyurethane, polystyrene-divinylbenzene copolymer), silicone rubber, quartz, latex, a transition metal, magnetic materials, silicon dioxide, silicon nitride, gallium arsenide, and derivatives thereof. Except for the reactive sites on the surface, substrate materials are generally resistant to the variety of chemical reaction conditions to which they may be subjected. The substrate useful in the present invention can be smooth or roughened. A smooth surface is one having a minimum of features on the surface that lead to roughness. A roughened surface is one that has a multitude of features on the surface that create friction. The substrate of the present invention includes a first substrate and a second substrate. The substrate can be flat or non-flat, flexible or rigid. In addition, the substrate can be porous, mesh or fabric. The substrate can be opaque or transparent, and can transmit or reflect light, or both. One of skill in the art will appreciate that other substrates are useful in the present invention.

“Electrode” refers to an electrical conductor for making contact with a part of a circuit. A circuit can often include several electrodes working together, such as the working electrode, the counter electrode and the reference electrode. The working electrode is the electrode in the system where the reaction or interaction of interest takes place. The counter electrode assists the working electrode in the measurement taking place. The reference electrode provides a stable electrode potential against which the potential of the working and counter electrodes are measured.

“Membrane” refers to a layer that allows material and fluids to move from one location to another through the membrane. The membrane can prevent some material from moving due to size limitations of the pores. When the pores are nanometer sized, the membrane can be a nanoporous membrane.

“Contacting” refers to bringing into close proximity at least two distinct objects such that a portion of at least one surface of the first object comes into contact with a portion of at least one surface of the second object.

“Covalently bound” refers to the formation of a covalent bond between the substrate of the present invention and a portion of the hydrogel.

“Biorecognition element” refers to a biological agent capable of specifically binding to another biological agent.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′)₂, Fab, Fv and rIgG. See also, Pierce Catalog and Handbook, 1994-1995 (Pierce Chemical Co., Rockford, Ill.). See also, e.g., Kuby, J., Immunology, 3^{rd} Ed., W.H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol: 5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

An antibody immunologically reactive with a particular antigen can be generated by recombinant methods such as selection of libraries of recombinant antibodies in phage or similar vectors, see, e.g., Huse et al., Science 246:1275-1281 (1989); Ward et al., Nature 341:544-546 (1989); and Vaughan et al., Nature Biotech. 14:309-314 (1996), or by immunizing an animal with the antigen or with DNA encoding the antigen.

Typically, an immunoglobulin has a heavy and light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs”. The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

References to “V_{H}” or a “VH” refer to the variable region of an immunoglobulin heavy chain of an antibody, including the heavy chain of an Fv, scFv, or Fab. References to “V_{L}” or a “VL” refer to the variable region of an immunoglobulin light chain, including the light chain of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

“Enzyme” refers to a biological catalyst.

“Aptamer” refers to oligonucleic acids or peptides that can bind to a specific target molecule.

“3,4-ethylenedioxythiophene (EDOT)” refers to the chemical structure below:

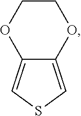

“2,3-dihydrothieno[3,4-b][1,4]dioxine-2-carboxylic acid (EDOT-COOH)” refers to the chemical structure below:

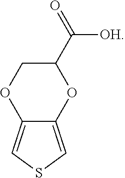 [SMILES: O=C(O)C1COc2cscc2O1]

“PEDOT random copolymer” refers to a copolymer of poly(3,4-ethylenedioxythiophene) (PEDOT) where EDOT and EDOT-COOH are copolymerized together to form PEDOT having EDOT-COOH randomly distributed in the copolymer:

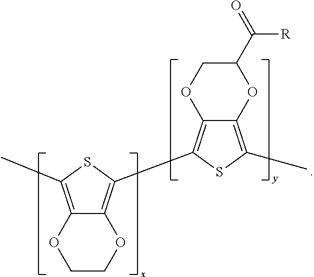

“Self-assembled monolayer” refers to a single layer of a substance such as an organic compound, assembled on the surface of a substrate. Self-assembled monolayers can be formed from a variety of materials, such as silanes on silicon or thiols on gold.

“Redox reporting moiety” refers to any moiety capable of generating a measurable signal in response to a change in the electrical potential. The measurable change in the electrical current can be correlated to concentration of the target analyte.

“Thiol moiety” refers to the group “—SH”.

“PEG” refers to polyethylene glycol and polyethylene oxide polymers. PEG can have any suitable molecular weight from less than 1,000 Daltons to over 100,000 Daltons. PEG can also be functionalized with a variety of groups at either the alpha or omega end. For example, PEG can be functionalized with one or more acrylate groups. When PEG is functionalized with two acrylate groups, PEG-diacrylate is formed. PEG can form a hydrogel by the crosslinking of PEG chains and the incorporation of water into the crosslinked polymer matrix.

As used herein, the term “hydrogel” refers to a highly-interdependent and interconnected, biphasic matrix having a solid component (usually a polymer, and more commonly a highly cross-linked polymer) that has both hydrophilic and hydrophobic character. Additionally, the matrix has a liquid component (e.g., water) that is retained in the matrix by intermolecular forces. The hydrophobic character provides the matrix with a degree of water insolubility while the hydrophilic character affords water permeability. One of skill in the art will appreciate that several different types of polymers can be used in combination to form hydrogels useful in the methods of the present invention.

“Microtiter well plate” refers to a plate having a plurality of wells for testing purposes, where the electrode is at the bottom of each well. Each electrode can be surrounded by a well perimeter defining walls around the electrode. Each well can be covered by a well cover.

“Disease marker” refers to a measurable indicator of the presence or relative risk of a particular disease. The disease marker can be genetic, or indicated by the relative concentration of a component in the patient's blood or body relative to the normal concentration. The disease marker can also be the relative function of a particular organ or process in the body of the patient, relative to the normal function of the organ or process.

“Biological sample” refers to any sample of the patient's body, including tissue, organ, fluid, etc.

“Peak reduction current” refers to the current at the formal potential of an electrochemical species that is being reduced.

“Square wave voltammetry” refers to the wave form employed in electrochemistry whereby potential (voltage) is swept not in a linear fashion but in a saw-tooth manner. This potential sweep method minimizes capacitive currents and is preferred for sensitive measurements of small changes in current.

“Tuberculosis” refers to the disease caused by myobacteria such as myobacterium tuberculosis.

“Hepatitis C” refers to the disease caused by the Hepatitis C virus, a member of the family Flaviviridae.

“Interferon-gamma” and “IFN-γ” refer to a type II interferon that binds the type II IFN receptor, interfering with viral infection of host cells. IFN-γ regulates a variety of biological functions, such as antiviral responses, cell growth, immune response, and tumor suppression, and may mediate a variety of human diseases.

“Photoinitiator” refers to a compound that initiates a polymerization process after irradiation. The photoinitiator can generate acid (a photo-acid generator or PAG) or a radical, among other initiating species. The acid, radical, or other species, then initiates a polymerization.

“Electropolymerization conditions” refers to a polymerization process that is initiated electrically instead of chemically. The conditions for electropolymerization include application of an electrical potential to the pre-polymer mixture with sufficient current and/or charge to initiate the polymerization. The electropolymerization conditions can vary depending on the polymer being prepared.

“Embedded” refers to the intercalation of the conductive polymer in the nanoporous membrane where the conductive polymer is not covalently linked to the nanoporous membrane.

### III. Conductive Biosensors

The present invention provides a biosensor for the detection of biological agents that can indicate a particular disease.

In some embodiments, the present invention provides a sensor having a substrate; at least one electrode contacting the substrate; a nanoporous membrane covering the electrode; and a biorecognition element selected from the group consisting of a peptide, an antibody, an enzyme, and an aptamer, wherein the biorecognition element is covalently bound to the electrode, or covalently bound to a PEDOT random copolymer embedded within the nanoporous membrane, the PEDOT random copolymer having a structure according to Formula I:

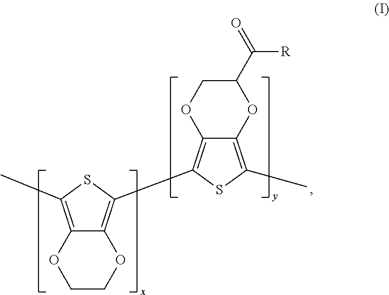

wherein each R is independently selected from the group consisting of —OH and the biorecognition element, wherein at least one R is the biorecognition element, and x and y are independently an integer of from about 1 to about 1000, wherein the sum of x and y is an integer of from about 2 to about 1000.

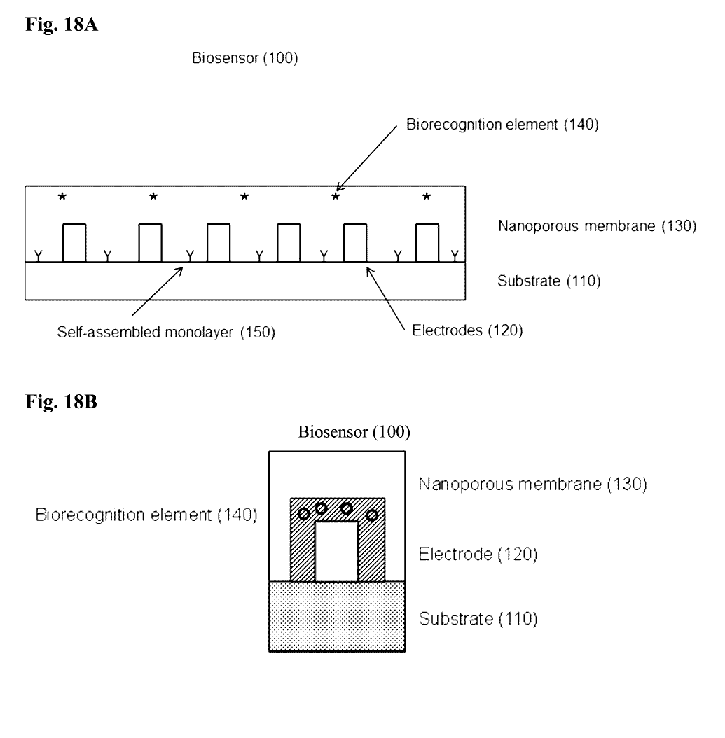

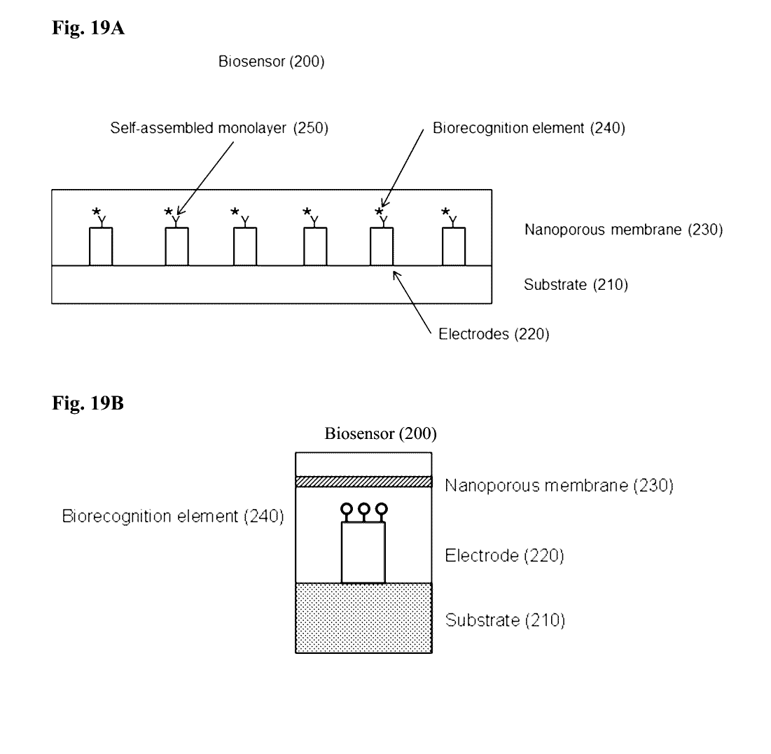

FIG. 18A-18B shows various embodiments of a biosensor (100) of the present invention in cross-section, including a substrate (110), electrodes (120), a nanoporous membrane (130) with biorecognition elements (140), and a self-assembled monolayer (150) on the substrate. FIG. 19A-19B also shows various embodiments of the biosensor (200) of the present invention in cross-section, including a substrate (210), electrodes (220), a nanoporous membrane (230), with biorecognition elements (240) linked to the electrodes via a self-assembled monolayer (250) on the electrodes. In some embodiments, the biorecognition element (240) can be linked directly to the electrode (220) without an intervening self-assembled monolayer (see FIG. 19B).

The substrate of the present invention can be any substrate suitable for supporting electrodes and binding to the PEG hydrogel. Examples of suitable substrates can be glass, insulators, ceramics, semi-conductors, metals, polymers, etc. Representative substrates include glass. In some embodiments, the substrate can be glass.

The biosensor of the present invention can include any suitable number and type of electrodes (120) contacting the substrate. For example, the biosensor can include a working electrode, a counter electrode and a reference electrode. The electrodes can be made of any suitable electrically conducting material. For example, the electrodes can be made of a metal such as, but not limited to, gold, silver, platinum, copper, etc. Other materials for the electrodes include metal salts such as silver chloride. In some embodiments, the biosensor includes a working electrode, a counter electrode and a reference electrode. In some embodiments, the electrodes are made of gold.

The electrodes of the present invention can be oriented in any suitable format relative to one another. For example, the electrodes can be circular with one electrode surrounded by another electrode. Moreover, the electrodes can be of any suitable shape, such as square, circular, or torus shaped.

In some embodiments, the electrode is a micropatterned gold electrode. In some embodiments, the sensor comprises a center working electrode, a surrounding counter electrode, and a reference electrode. In some embodiments, the working electrode is a gold electrode. In some embodiments, the reference electrode is a silver/silver chloride electrode.

The biosensor of the present invention can also include a nanoporous membrane covering the electrodes. The nanoporous membrane can be prepared from any suitable material. For example, the nanoporous membrane can be a polymer, an inorganic material such as a ceramic or oxide such as aluminum oxide, or other similar materials. The nanoporous membrane can be conductive or non-conductive. The nanoporous membrane can have pores of any suitable size, such as from 10 nm to 100 am. The nanoporous membrane can be of any suitable thickness, such as from 10 nm to 10 mm. In some embodiments, the nanoporous membrane can be aluminum oxide. In some embodiments, the biosensor of the present invention does not include a nanoporous membrane.

The nanoporous membrane can also be a conductive hydrogel. Any suitable conductive hydrogel can be used in the present invention. The hydrogel can be prepared from any suitable material, such as polyethyleneglycol (PEG). The PEG can be functionalized with crosslinkable groups such as acrylates, methacrylates or other polymerizable groups. The PEG can be any suitable molecular weight. For example, the PEG can have a molecular weight of about 1 kDs, 2, 3, 4, 5, 6, 7, 8, 9 or 10 kDa. PEG can also have a molecular weight up to about 100 kDa. In some embodiments, the PEG can be a PEG-diacrylate with a molecular weight of about 6 kDa.

In some embodiments, the PEG hydrogel can be covalently linked to the substrate. For example, the substrate can be modified with a self-assembled monolayer capable of covalently binding to the PEG-diacrylate during the polymerization process for preparing the PEG hydrogel. In some embodiments, the substrate is modified with a self-assembled monolayer containing an acrylate functional group. Covalent attachment of the nanoporous membrane to the substrate can reduce delamination of the nanoporous membrane and improve stability and life span of the biosensor of the present invention.

The conductive hydrogel can also include a conductive polymer. Any suitable conductive polymer matrix is suitable in the biosensor of the present invention. For example, the conductive polymer can include a polymer of poly(3,4-ethylenedioxythiophene) (PEDOT). Other conductive polymer are also useful in the present invention, including, but not limited to, polypyrroles, polythiophenes, etc.

The conductive hydrogel can be prepared by any suitable means. For example, the PEG hydrogel can be prepared first using vinyl polymerization methods, followed by preparation of the PEDOT polymer using electropolymerization methods. The vinyl polymerization methods include acid catalyzed or radical initiated polymerization. In some embodiments, polymerization of the PEG can be photoinitiated. The PEDOT polymer can be a copolymer of several EDOT monomer units, including EDOT and EDOT-COOH. When the EDOT monomers are copolymerized, a PEDOT random copolymer can be formed. The PEDOT copolymer can be any suitable molecular weight, with any suitable distribution of EDOT and EDOT-COOH comonomers. For example, the ratio of EDOT and EDOT-COOH comonomers can be from about 1000:1 to about 1:1000, or about 500:1, 100:1, 50:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:50, 1:100 or about 1:1000. Other ratios of the monomers are also useful.

In some embodiments, the PEDOT random copolymer can have the structure according to Formula I:

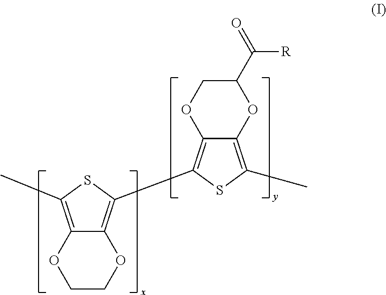

wherein each R is independently selected from the group consisting of —OH and the biorecognition element, wherein at least one R is the biorecognition element, and x and y are independently an integer of from about 1 to about 1000, wherein the sum of x and y is an integer of from about 2 to about 1000. In some embodiments, the ratio of x to y is from about 10:1 to about 1:10. In some embodiments, the ratio of x to y is about 1:4.

The PEDOT random copolymer can be prepared before or after preparation of the PEG hydrogel. In some embodiments, the PEDOT random copolymer is prepared after preparation of the PEG hydrogel by depositing the EDOT and EDOT-COOH monomers in the PEG hydrogel and exposing the PEG hydrogel to electropolymerization conductions such that the PEDOT random copolymer is prepared in the PEG hydrogel, but is not covalently bound to the PEG hydrogel.

The present invention includes any suitable biorecognition element. For example, the biorecognition element can include an antibody, an aptamer, a camelid, a peptide, a protein, or other biological agent. In some embodiments, the biorecognition element can be a peptide, an antibody, an enzyme or an aptamer. In some embodiments, the biorecognition element can be an antibody. In some embodiments, the biorecognition element can be an enzyme. In some embodiments, the biorecognition element can be an aptamer. In some embodiments, the biorecognition element can be IFN-γ. In some embodiments, the biorecognition element can be bovine-IFN-γ.

Any suitable aptamer or enzyme can be used as a biorecognition element in the present invention. Aptamers in the present invention may be specific to inflammatory cytokines including but not limited to TNF-α, IL-2, IL-4, IL-6, IL-10, IL-17, IL-21 and TGF-β. Aptamers may also be specific to cell surface markers including but not limited to CD4, CD8, CD36, CD 14, CD45 and EpCAM. Aptamers may also be specific to components of pathogens including but not limited to HIV, HBV, HCV and sexually transmitted diseases (STDs). Aptamers may also be specific to transmembrane proteins present in extracellular vesicles for example CD63. Any redox enzyme may be used as a biorecognition element in the present invention. Major groups of enzymes include but are not limited to oxidoreductases (e.g. peroxidase, glucose oxidase, lactate oxidase) and NADH-dependent enzymes.

Other biorecognition elements include peptides. The peptides can be any suitable peptide that bind to the disease marker, or serve as the substrate for proteases. Such a peptide allows the biosensor of the present invention to detect protease activity.

The biorecognition element can be linked to the conductive polymer, or directly to the electrodes. In some embodiments, the biorecognition element can be linked to the conductive polymer. When the conductive polymer is the PEDOT random copolymer, the biorecognition element can be linked to the carboxylic acid group of the PEDOT random copolymer. In some embodiments, the biorecognition element is covalently bound to the PEDOT random copolymer embedded within the nanoporous membrane, wherein the nanoporous membrane comprises a PEG hydrogel having PEG chains with a molecular weight of from about 1000 Da to about 10,000 Da.

In some embodiments, the biorecognition element can be covalently linked to the electrodes. When the biorecognition element is covalently linked to the electrodes, the electrodes can be modified with a self-assembled monolayer capable of covalently linking to the biorecognition element. In some embodiments, the biorecognition element is covalently linked to the electrode to form a self-assembled monolayer of the biorecognition element on the electrode. In some embodiments, the biorecognition element is an aptamer having a redox reporting moiety and a thiol moiety, wherein the thiol moiety is covalently bound to a gold working electrode.

The biosensor of the present invention can have one biorecognition element or several different biorecognition elements. For example, the PEDOT conductive polymer can be functionalized with several different biorecognition elements. This allows for the simultaneous detection of several different disease markers, as well as greater flexibility when testing for one of several different disease markers. Similarly, when the biorecognition element is linked directly to the electrode, each electrode can be modified with one biorecognition element or several different biorecognition elements. See, for example, Liu et al., “Detecting multiple cell-secreted cytokines from the same aptamer-functionalized electrode” Biosensors and Bioelectronics 2015, 64, 43-50, incorporated herein in its entirety.

The biosensor of the present invention can include a variety of other components. For example, the biosensor can include a microtiter well plate, a well cover, among others. In some embodiments, the biosensor also includes a microtiter well plate for housing the biosensor. In some embodiments, the biosensor also includes a well cover comprising one or more walls contacting the substrate, the walls defining a well perimeter surrounding the electrode.

### IV. Methods for Detecting Disease Markers

The present invention also provides methods for using the biosensor of the present invention for disease detection by detecting disease markers. In some embodiments, the present invention provides a method for detecting a disease marker in a biological sample comprising contacting a sensor of the present invention with the biological sample and detecting the binding of the disease marker to a biorecognition element, thereby detecting the disease marker.

The detecting can be performed by any suitable means or device. For example, the detecting can involve detecting a change in the electrical signal generated by the biosensor of the present invention. Without being bound by theory, when the disease marker binds to the biorecognition element, a change in the electrical signal of the nanoporous membrane is generated and transmitted from the biorecognition element to the electrode via the conductive polymer.

In some embodiments, the detecting the binding of the disease marker to the biorecognition element comprises measuring the peak reduction current of the PEDOT random copolymer. In some embodiments, the binding of the disease marker to the biorecognition element is detected using square wave voltammetry.

Any suitable disease marker can be used for detection. For example, the disease marker can be a peptide, protein, antibody, enzyme, cell, tissue, etc. from the patient. In some embodiments, the disease marker can be interferon. In some embodiments, the disease marker can be interferon-gamma. Other disease markers include exosomes, extracellular vesicles (EVs). The extracellular vesicles contain transmembrane proteins that can be detected using specific aptamers. Exemplary transmembrane proteins include, but are not limited to, CD63. Other disease markers include proteases such as, but not limited to, matrix metalloproteinases (MMPs) MMP2, MMP4, and MMP9, as well as urokinase (uPA).

The disease marker can indicate the presence of any disease, such as cancer, viral infection, bacterial infection, heart disease, autoimmune disorders, etc. Representative viral infections include, but are not limited to, HIV, HBV, HCV, herpes, Varicella-zoster, Epstein-barr, cytomegalovirus, papillomavirus, BK virus, JC virus, smallpox, coxsackievirus, polio, rhino virus, SARS, yellow fever, dengue, West Nile, Rubella, influenza, Ebola, Marburg, measles, mumps, respiratory syncytial, metapneumovirus, rabies, and others. Representative bacterial infections include, but are not limited to, tuberculosis, tetanus, typhoid fever, diphtheria, syphilis and leprosy. Representative cancers include, but are not limited to, leukemia, CNS, renal, non-small cell lung cancer, melanoma, prostate, kidney, liver, breast, ovarian, pancreatic or colorectal cancer. Other diseases

In some embodiments, the disease marker is indicative of an infection by tuberculosis or hepatitis C. In some embodiments, the disease marker comprises IFN-γ. In some embodiments, the biorecognition element comprises an interferon-gamma (IFN-γ) antibody. In some embodiments, the biorecognition element comprises an aptamer specific for IFN-γ.

### V. Conductive Hydrogels

The present invention also provides conductive hydrogels for use in the biosensors of the present invention. In some embodiments, the present invention provides a conductive hydrogel including a covalently cross-linked poly(ethylene glycol) (PEG) hydrogel; and a poly(3,4-ethylenedioxythiophene) (PEDOT) random copolymer embedded within the PEG hydrogel, the PEDOT random copolymer having a structure according to Formula I:

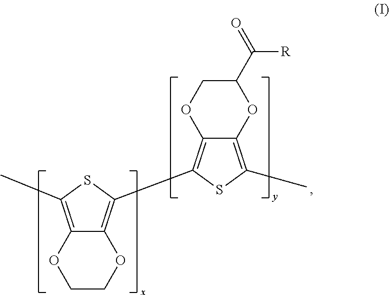

wherein each R is independently selected from the group consisting of —OH and a biorecognition element selected from the group consisting of a peptide, an antibody, an enzyme, and an aptamer, wherein at least one R is the biorecognition element, and x and y are independently an integer of from about 1 to about 1000, wherein the sum of x and y is an integer of from about 2 to about 1000.

In some embodiments, the PEG hydrogel comprises PEG chains having molecular weights of from about 1000 Da to about 10,000 Da. In some embodiments, the PEG hydrogel comprises PEG chains having a molecular weight of about 6000 Da. In some embodiments, the ratio of x to y is from about 10:1 to about 1:10. In some embodiments, the ratio of x to y is about 1:4. In some embodiments, the covalently cross-linked poly(ethylene glycol) hydrogel is prepared from PEG-diacrylate.

### VI. Methods for Preparing Conductive Hydrogel Biosensors

The conductive hydrogels of the present invention can be prepared by any means known to one of skill in the art. For example, a PEG hydrogel can be prepared first, and then a conductive polymer, such as PEDOT, can be polymerized within the PEG hydrogel so that the conductive polymer is embedded in the PEG hydrogel but not covalently linked to the PEG hydrogel.

In some embodiments, the present invention provides a method for preparing the conductive hydrogel of the present invention, including contacting a PEG-diacrylate and a photoinitiator under polymerization conditions suitable to form a PEG hydrogel; contacting the PEG hydrogel with a solution comprising 3,4-ethylenedioxythiophene (EDOT) and 2,3-dihydrothieno[3,4-b][1,4]dioxine-2-carboxylic acid (EDOT-COOH) under electropolymerization conditions sufficient to form a poly(3,4-ethylenedioxythiophene) (PEDOT) random copolymer of Formula I embedded within the PEG hydrogel:

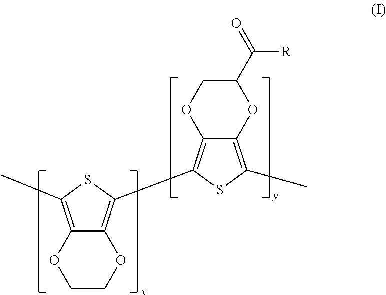

wherein each R is —OH, and x and y are each an integer of from about 1 to about 1000, wherein the sum of x and y is an integer of from about 2 to about 1000; and contacting the hydrogel with a biorecognition element under conditions sufficient to covalently bind the biorecognition element to the PEDOT random copolymer, thereby forming the PEDOT random copolymer of Formula I wherein at least one R is a biorecognition element selected from the group consisting of a peptide, an antibody, and an aptamer, thereby preparing the conductive hydrogel of the present invention.

The PEG-diacrylate can be any suitable PEG-diacrylate having the formula:

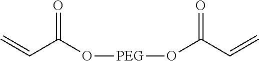

The PEG portion of the PEG-diacrylate can have any suitable molecular weight, as described above. In some embodiments, the PEG-diacrylate has a molecular weight of from about 1000 Da to about 10,000 Da. In some embodiments, the PEG-diacrylate has a molecular weight of about 6000 Da.

The PEG hydrogel can be prepared by any means known to one of skill in the art. For example, the PEG can be functionalized at one or both ends with a suitable polymerizable group. Suitable polymerizable groups include, but are not limited to, acrylates, methacrylates, styrenics, cyanoacrylates, and others. The polymerization of the PEG hydrogel can be performed under radical polymerization conditions, or acid or base polymerization conditions. In some embodiments, the polymerization is performed under radical polymerization conditions. In some embodiments, the polymerization conditions for forming the PEG hydrogel comprises irradiating the PEG-diacrylate.

When the PEG hydrogel is prepared by irradiating the PEG-diacrylate with a photoinitiator, the irradiation can be performed under a variety of conditions. For example, the PEG hydrogel can be irradiated for from 1 second to more than 1 minute, including 2, 3, 4, 5, 10, 15, 20, 30, 40, and 50 seconds. Irradiation for longer periods is also contemplated by the present invention. The wavelength and intensify of irradiation can depend on the photoinitiator used, the concentration of photoinitiator, and the degree of crosslinking desired in the PEG hydrogel.

The photoinitiator can be any suitable photoinitiator known in the art. For example, the photoinitiator can be azobisisobutyronitrile, benzoyl peroxide, camphorquinone, or 1-[4-(2-Hydroxyethoxy)-phenyl]-2-hydroxy-2-methyl-1-propane-1-one (IRGACURE® 2959 from Ciba).

The conductive polymer can be polymerized by any means known to one of skill in the art, such as electropolymerization. As discussed above, the conductive polymer can be a single polymer or a copolymer. Representative polymers including polythiophenes, polypyrroles, and polyphenylenes. When the conductive polymer is a polythiophene based polymer, any suitable thiophene-based monomer can be used. Representative thiophene monomers include, but are not limited to, thiophene, 3-methylthiophene, 3,4-ethylenedioxythiophene (EDOT), and 2,3-dihydrothieno[3,4-b][1,4]dioxine-2-carboxylic acid (EDOT-COOH). The EDOT-COOH monomer includes a carboxylic acid functional group that can be functionalized with the biorecognition element.

In some embodiments, the conductive polymer is a polythiophene polymer. In some embodiments, the conductive polymer is a PEDOT copolymer. When the conductive polymer is a PEDOT copolymer, the EDOT and EDOT-COOH monomers can be present in any suitable ratio, as described above. In some embodiments, the EDOT and EDOT-COOH are present in a ratio of from about 10:1 to about 1:10. In some embodiments, the EDOT and EDOT-COOH are present in a ratio of about 1:4.

The conductive polymer can be modified with the biorecognition element using any methods known to one of skill in the art. For example, when the conductive polymer is a PEDOT copolymer using EDOT-COOH, the biorecognition element can be linked to the PEDOT copolymer using amide formation chemistry. Preparation of activated esters, such as via EDC chemistry, are well known and useful in the present invention.

The biorecognition element can be any suitable agent as described above. In some embodiments, biorecognition element is an antibody. In some embodiments, the biorecognition element is an aptamer.

### VII. Examples

### Example 1

### Preparation of PEDOT-PEG Hydrogel

We constructed a novel composite conducting hydrogel constituting conductive carboxyl-functionalized PEDOT and a high molecular weight PEG for development of an immunosensor. The carboxyl functionality provides capability of bioconjugation. The bio-interfaces were prepared by polymerizing PEG on top of miniaturized Au electrodes followed by electro-polymerizing PEDOT copolymer into the porous PEG gel using aqueous microemulsion. The PEDOT/PEG copolymer was compositionally tunable and controlled to deposit on an array of electrode surfaces. The COOH groups present on the conducting polymer chain were tailored with B-IFN-γ antibodies via active ester groups. The antibody immobilized conducting hydrogel electrodes were utilized for label-free electrochemical detection of B-IFN-γ in buffer and bovine plasma. Binding of B-IFN-γ to antibody results in electrochemical signal decrease caused due hindrance of electron transfer. This signal-off sensing method does not need any external redox labels for detection.

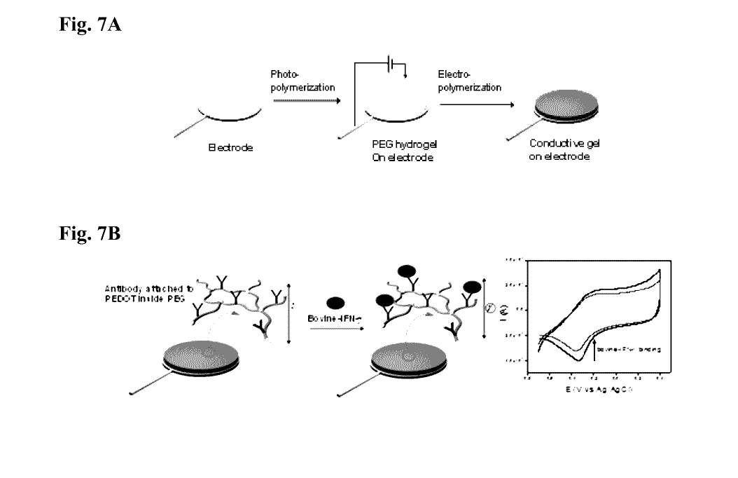

The process of fabricating sensing surfaces begins by photopolymerizing PEG hydrogel on top of electrodes. Subsequently, PEDOT copolymer is electropolymerized within PEG hydrogel. The electrodes deposited with conductive hydrogel showed faint blue when it is oxidized and turned dark blue when it is reduced due to its intrinsic properties (FIG. 7A). Scanning electron microscopy (SEM) was used to investigate the morphological features of conducting polymer and conducting hydrogel surfaces. As seen from SEM images (FIG. 7B), the PEDOT deposition on a gold electrode showed rough granular morphology and UV polymerized PEG hydrogel showed porous morphology. The electropolymerization of PEDOT inside PEG fills the pores in PEG. Sole PEDOT polymerization on Au surfaces suffered from poor mechanical performances.

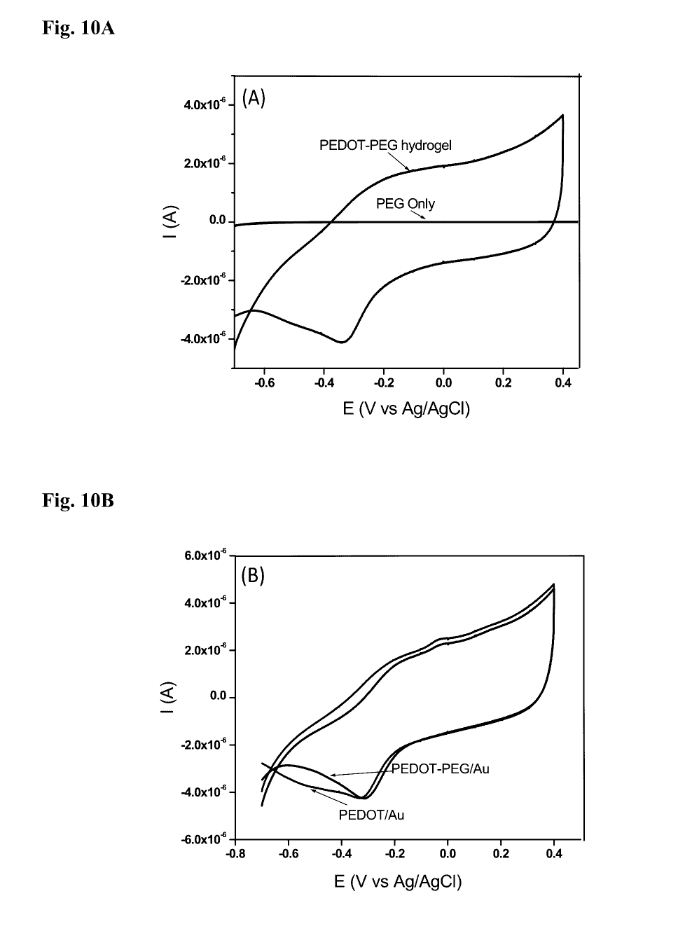

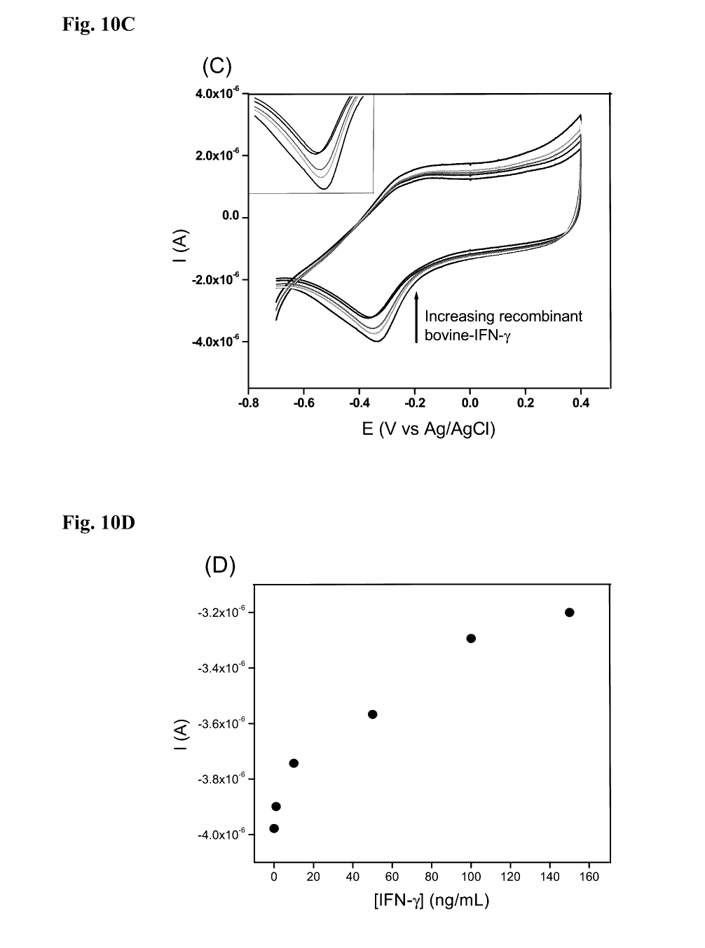

Importantly, PEDOT molecules within the gel undergo redox reactions upon application of voltage and possess characteristic redox peaks. The cyclic voltammograms of PEG hydrogel without and with incorporation of conductive PEDOT revealed enhanced electrical properties of conductive hydrogel over the regular PEG hydrogel (FIG. 10A). The incorporation of conducting polymer in PEG showed greater redox current values by almost 100 fold. The sharp intrinsic oxidation and reduction property of PEDOT was further utilized for the biosensing application. PEDOT/PEG hydrogel showed similar CV characteristic to PEDOT which proves that PEG did not affect the electrochemical behavior of PEDOT (FIG. 10B).

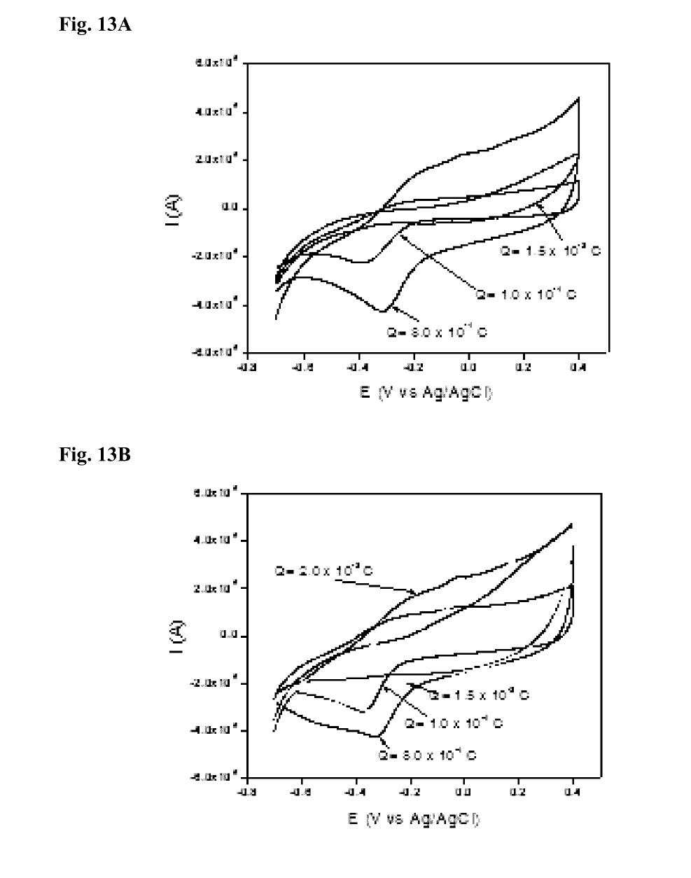

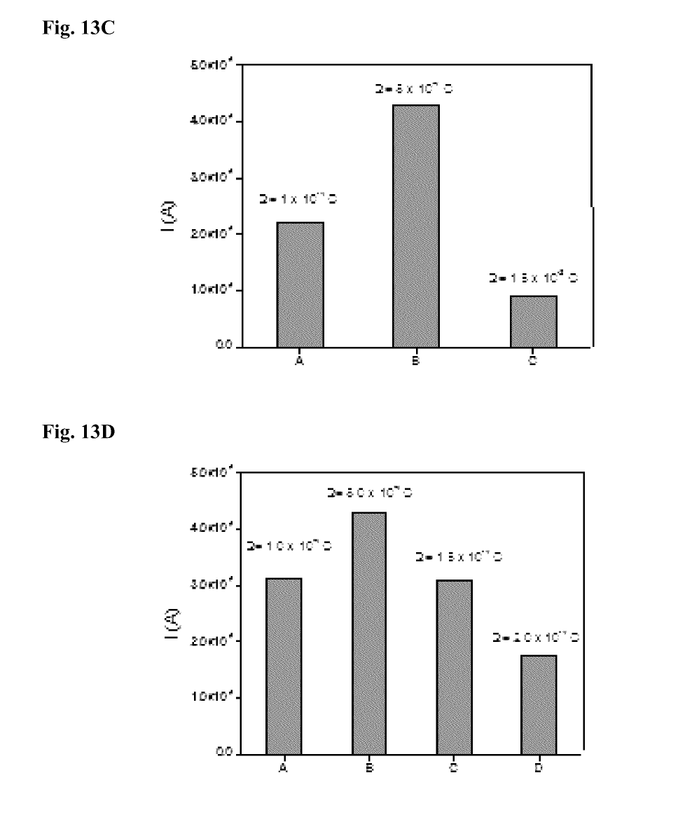

The deposition of PEDOT inside the PEG gel was optimized by varying the applied charges for the electropolymerization. As seen from the data of redox behavior as a function of charge deposited, we could confirm that the enhanced redox properties at the charge of 8×10⁻⁴ C, while the lower and higher charge (or thickness of PEDOT film) leads to poor redox peaks and reduction current (FIG. 13A-13D).

A pre-polymer PEG hydrogel solution, 5% PEG-diacrylate and 1% photoinitiator (Irgacure 2959) in PBS was coated onto the Au electrodes of the patterned slides. These slides were exposed to UV radiation (60 mJ/cm²) for 5 s through an aligned photo mask on top of the electrodes. The prepared PEG hydrogel on top of patterned Au electrodes was then incubated in the aqueous solution of 10 mM of EDOT-COOH containing 2.5 mM EDOT, 0.05 M SDS and 0.1 M LiClO₄ in the batch mode setup for electropolymerization. This monomer solution was electropolymerized into the porous PEG hydrogel amperometrically at a constant potential of 1.1 V using CHI 6044d Electrochemical Analyzer (CH Instruments, Inc. Austin, Tex.). The resulting polymer has enhanced redox properties at the charge of 8×10⁻⁴ C of the polymer film.

Antibody is attached covalently via carboxylic groups present on PEDOT. Carboxylic group was activated to immobilize antibody covalently. The conducting polymer hydrogel array was incubated with 0.2 mM EDC and 0.05 mM NHS in DI water for 15 min. The array was rinsed with PBS and incubated with 50 μg/mL bovine INF-γ antibody in PBS overnight at 4° C. The electrochemical measurement was performed on conducting polymer hydrogel array using CHI instrument. Cyclic voltammetry was employed in the range between −0.7V and 0.4V to measure binding of bovine INF-γ. Peak current (Iₚ) was measured at −0.35V during each measurement and the signals were plotted by a function of concentration versus current values.

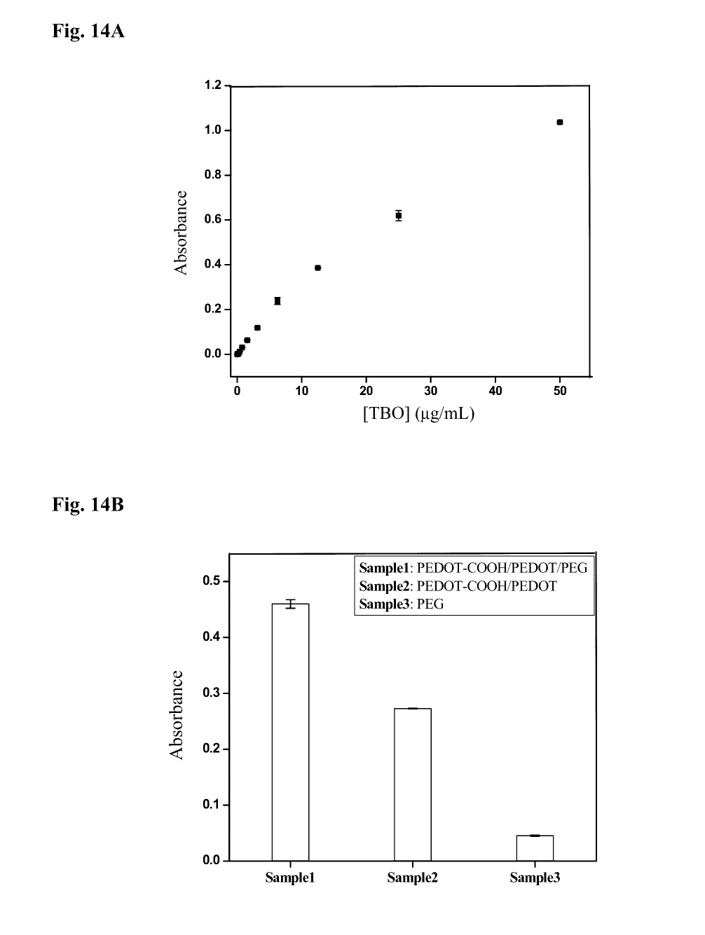

The COOH groups present on the conducting polymer are activated by standard EDC-NHS chemistry in order to immobilize B-IFN-γ antibody molecules. A mixture of EDOT-COOH and EDOT in a LiClO₄ aqueous solution containing sodium dodecyl sulfate (SDS) was polymerized amperometrically within PEG hydrogel layer at potential 1.1 V keeping a constant charge of 8×10⁻⁴ C. This process yielded an interconnected polymer structure inside the PEG hydrogel. The number of COOH groups were quantified by toluidine blue O (TBO) staining.^{[24-25]} The calibration curve for COOH groups TBO absorption at 633 nm was obtained (FIG. 14A-14B) the density of the COOH groups was estimated to be 3.6×10¹⁶ molecules/cm² which was ca. 2-fold higher than the density of COOH found in electrochemically deposited PEDOT-COOH/PEDOT on bare gold surface (1.7×10¹⁶ molecules/cm²).

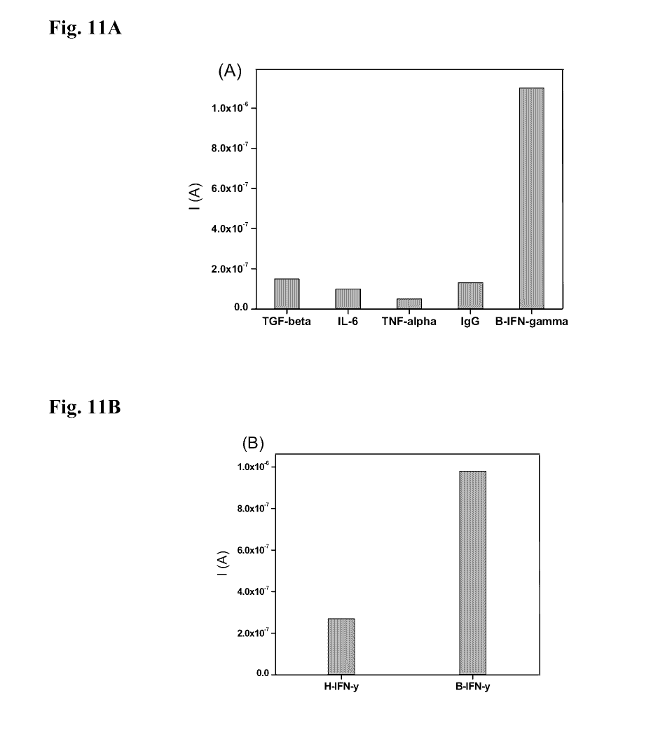

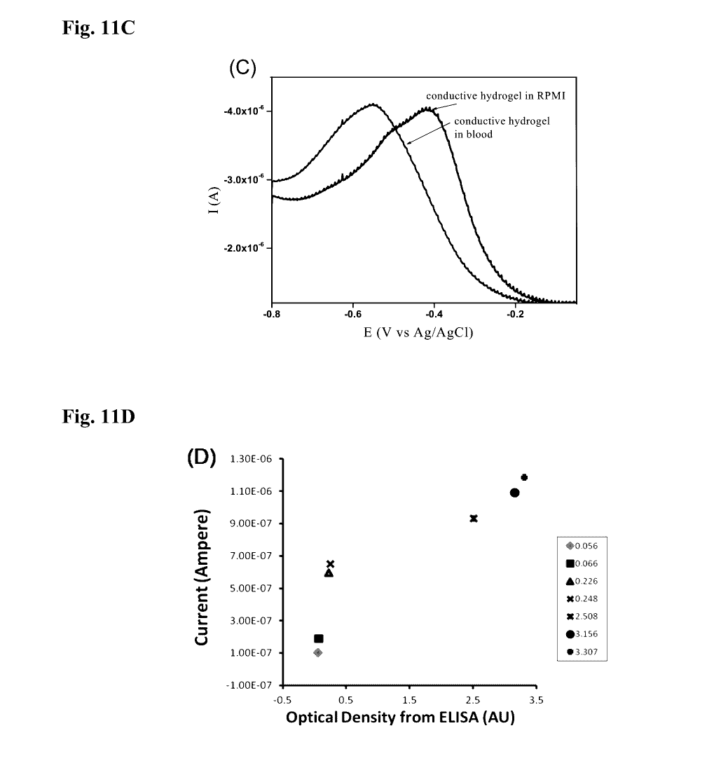

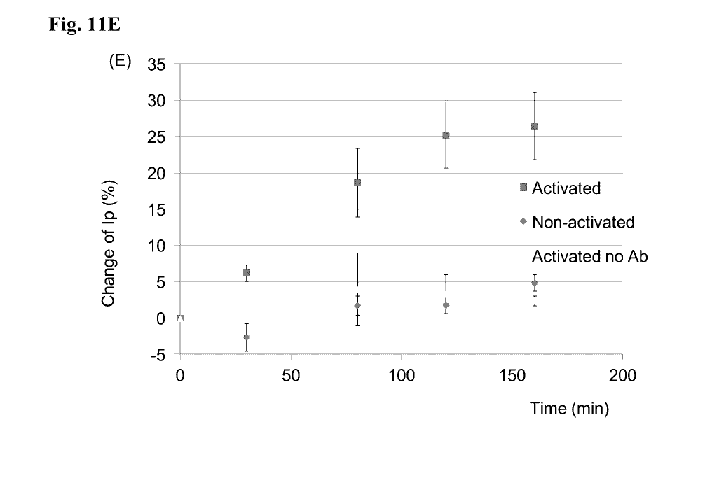

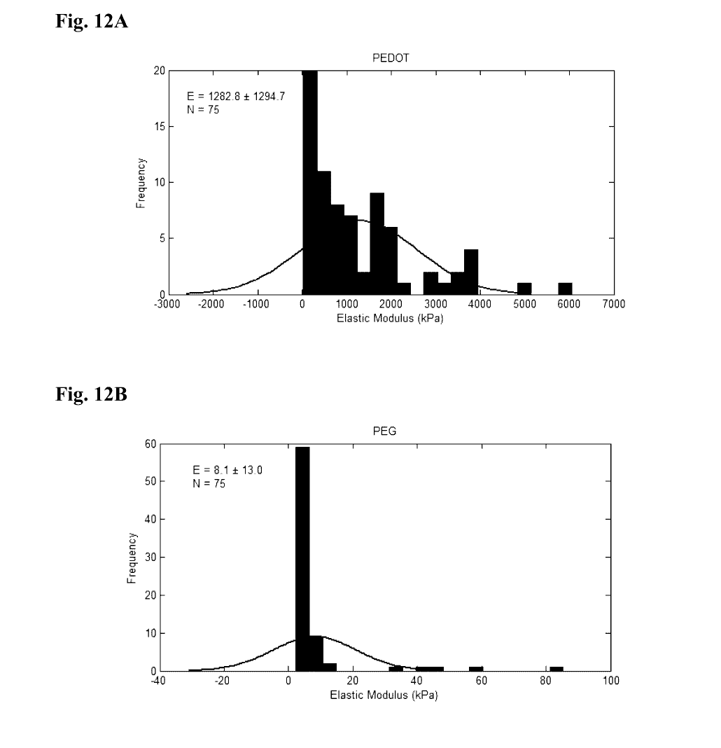

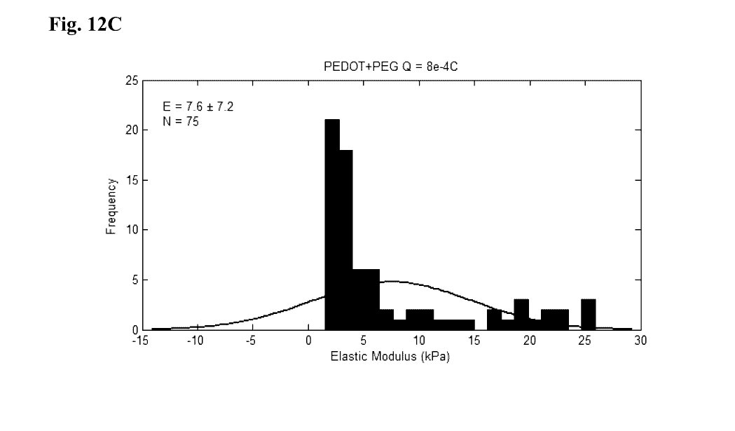

Anti-bovine IFN-γ antibody was immobilized covalently on carboxylated PEDOT chains inside the conducting polymer hydrogel using EDC-NHS. Response of antibody immobilized conducting polymer hydrogel was studied in PBS using cyclic voltammetry. This highly conducting hydrogel matrix gives response even in PBS and obviates the need of external redox indicators. The antibody immobilized conducting polymer hydrogel surface was challenged with different concentrations of recombinant B-IFN-γ and the cyclic voltammograms were recorded. Capture of B-IFN-γ by antibody-containing gel hinders electron transfer through the polymer chain and causes the redox peak to decrease (FIG. 11C). The drop in the PEDOT reduction peak in response to different concentration of B-IFN-γ forms the basis of electrochemical sensing of this analyte.

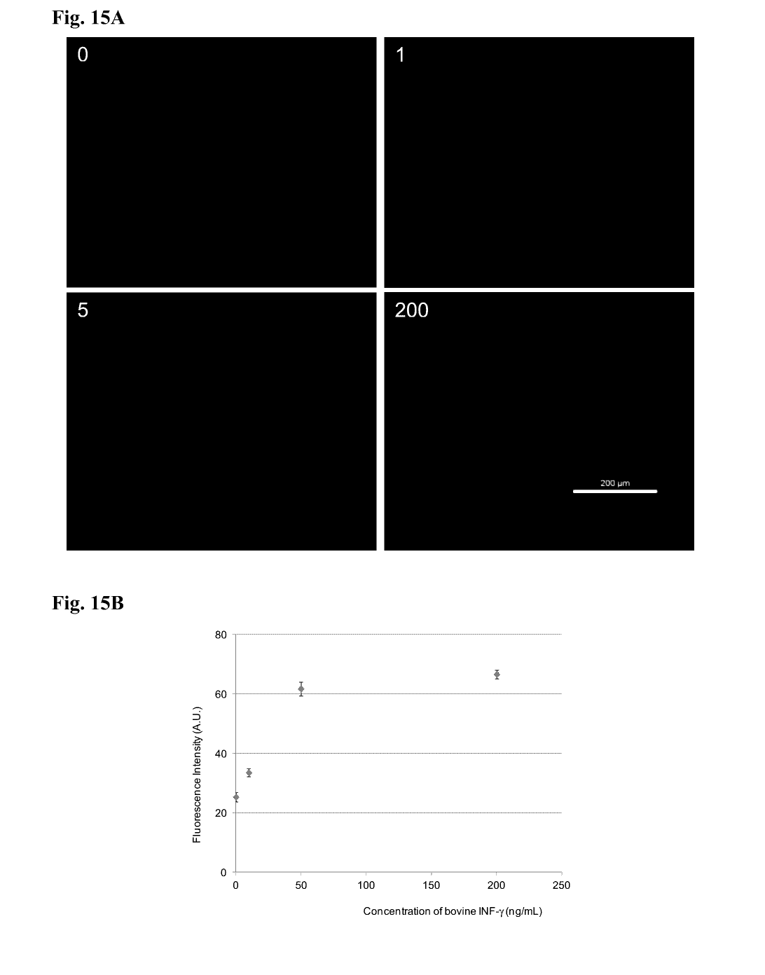

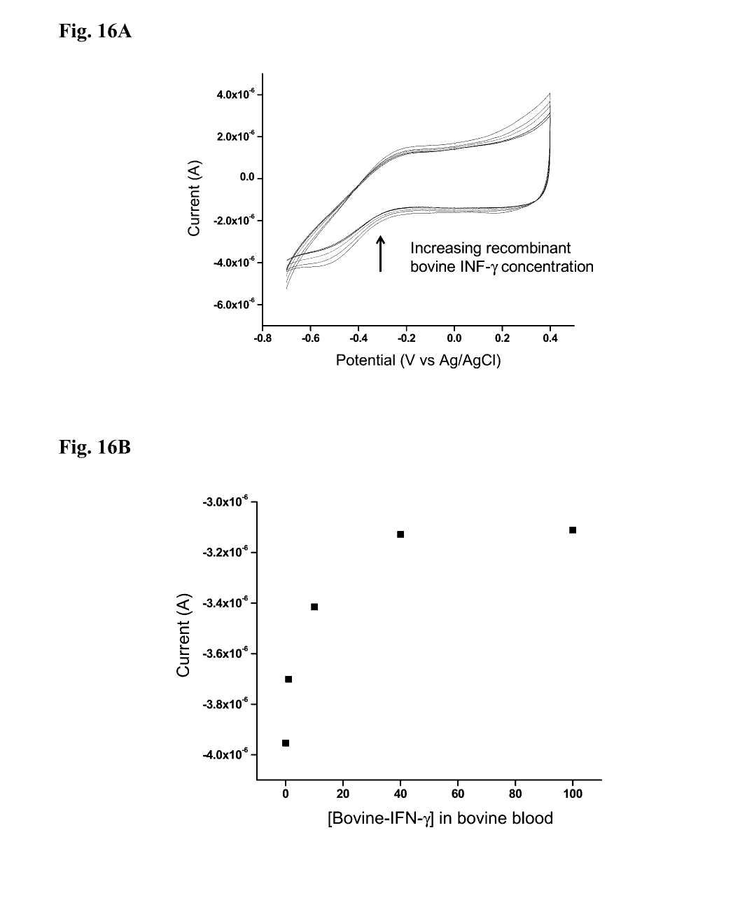

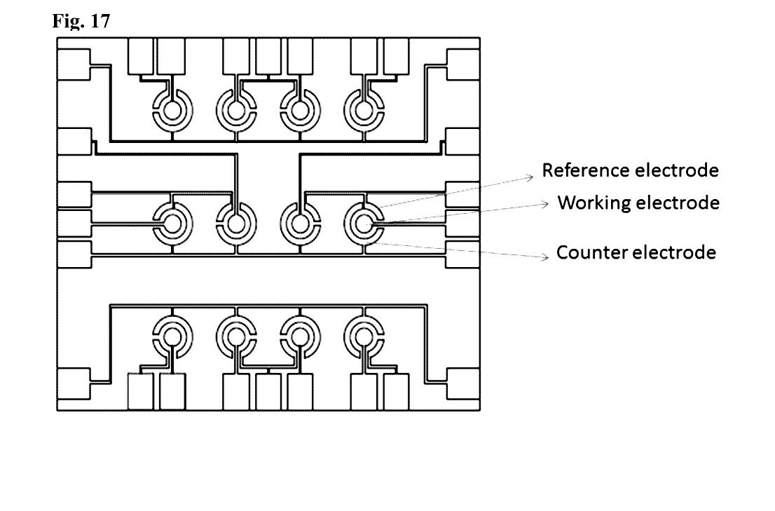

We obtained a calibration curve for recombinant B-IFN-γ concentration versus reduction current of antibody functionalized conducting hydrogel on electrodes (FIG. 11D). We believe that the binding of electrochemically insulating target molecule to the antibody functionalized PEDOT chains hinders the charge transfer properties and conductivity of PEDOT that results in suppression of reduction current. Additionally, sandwich assays were performed on ITO electrodes to visualize the immobilized B-IFN-γ. The electrodes were then incubated with recombinant B-IFN-γ followed by biotinylated secondary antibody and fluorophore-conjugated streptavidin. The fluorescence was measured using a fluorescent microscope. Incubation with fluorescently labeled antibody revealed the binding of recombinant B-IFN-γ to the antibody immobilized conductive hydrogel (FIG. 15A: fluorescence images).

### Example 2

### Preparation of Sensor

Gold and ITO patterned electrodes were prepared using photolithography and wet-etching approaches as previously reported. The micropatterned glass slides, containing photoresist on top of Au surface, were treated with oxygen plasma for 10 min and incubated in 0.05% solution of (3-acryloxypropyl) trichlorosilane in toluene for about one hour under nitrogen atmosphere to obtain a self-assembled monolayer of silane on the glass regions. The procedure is described in more detail below.

Positive resist (S1813) was spin-coated (2000 rpm for 30 sec) on glass slides coated with 15 nm Cr adhesion layer and 100 nm Au layer resulting in formation of a thick layer of photoresist. The photoresist-coated glass slides were soft-baked on a hot-plate at 115° C. for 1 min, then placed in contact with a photomask and exposed to 365 nm UV source. The substrates were then placed into a developer solution (MF 319). After development step, Au-coated glass slides were immersed in Au etching solution followed by immersion in Cr etching solution. Metal was selectively removed from the regions not protected by photoresist, resulting in formation of Au micropatterns of diameter 1500 am. Importantly, the photoresist layer on top of Au microelectrodes was not removed immediately after etching but was employed to protect underlying Au regions during the silane modification protocol so as not to lose conductivity.

The substrates with photoresist-covered Au electrodes were modified with 3-(acryloxypropyl) tricholosilane. After silane modification, substrates were sonicated in acetone to remove photoresist.

The nanoporous membrane including the PEDOT-PEG hydrogel described in Example 1 is then prepared on top of the electrodes and becomes attached to the substrate. The biorecognition element is then attached to the PEDOT-PEG hydrogel, again, following the procedure in Example 1.

### Example 3

### Detection of Tuberculosis

The antibody (bovine-IFN-γ) functionalized PEDOT conducting hydrogel was placed in the electrochemical cell set up of Example 2. Different concentrations of target (recombinant interferon gamma) prepared in 1×PBS were introduced into the electrochemical cell and incubated for about 20 mins. The cyclic voltamograms were recorded.

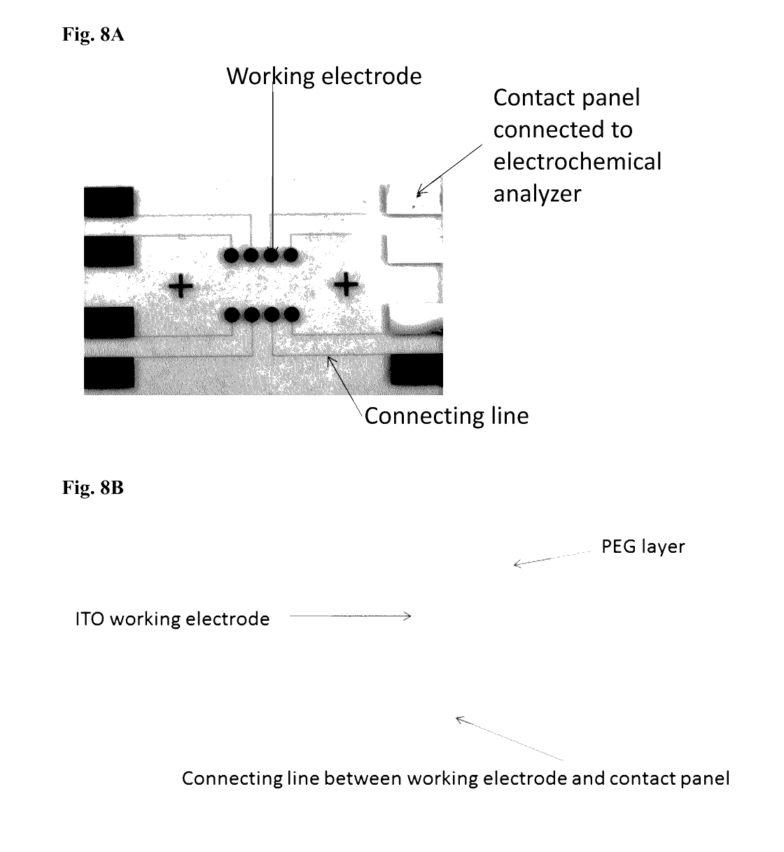

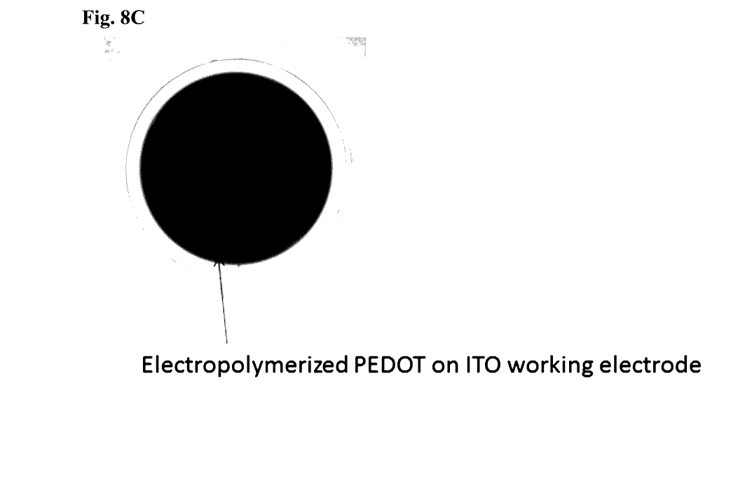

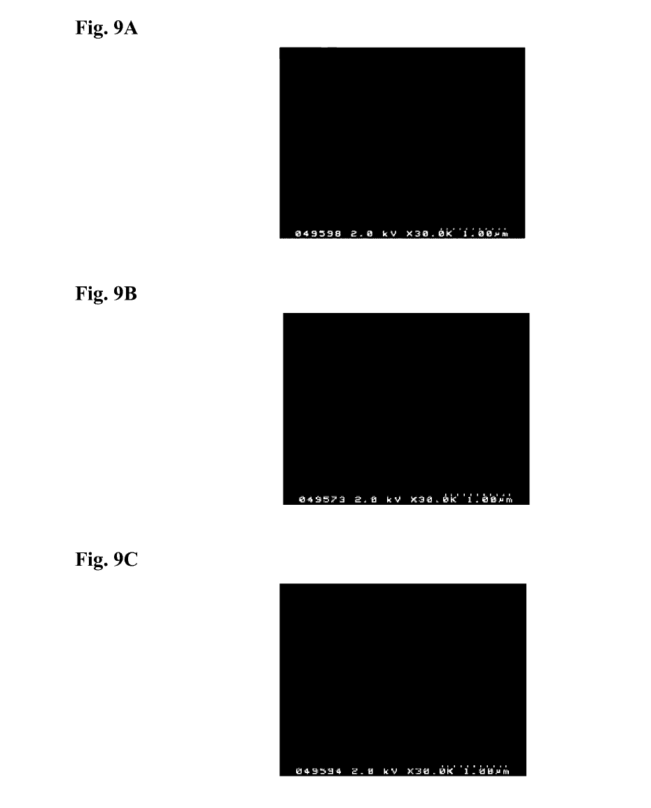

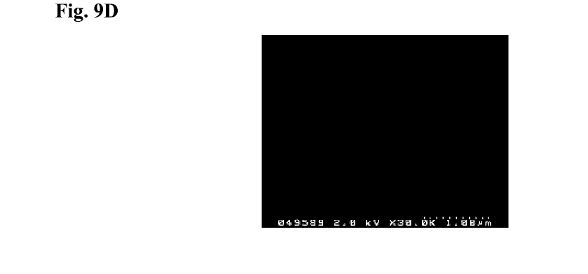

Each sensing electrode (antibody functionalized conductive hydrogel on Au microelectrode) was connected to potentiostat via Au contact pads as shown in FIG. 8A. Each sensing electrodes was individually addressed by potentiostat in this way.

The Au electrodes having conductive gel on the sensing chip were working electrodes and were individually addressed by potentiaostat. We used external Ag/AgCl reference and platinum wire counter electrodes. These were dipped in the lectrolyte solution of the electrochemical cell set up as shown in Fig. and were connected to potentiostat.

To test the specificity of the conducting hydrogel bioelectrodes, the sensor was challenged with nonspecific cytokines and proteins including TGF-β, IL-6, TNF-α, and IgG. We observed minimal signal reduction with the nonspecific cytokines (FIG. 11A) and human IFN-γ (FIG. 11B). This result demonstrates that our sensor can detect B-IFN-γ in a mixture of nonspecific cytokines. To demonstrate feasibility of our sensor to cell-related experiments, we wanted to detect signal responses in serum-containing media and whole blood. As shown in FIG. 11C, addition of whole blood resulted in negligible signal loss as compared to RPMI media alone. The antibody immobilized conductive hydrogel sensor was challenged with the known B-IFN-γ spiked in (1:1) TB-free bovine blood/PBS (FIG. 16A-16B). The addition of bovine blood resulted in signal loss of ˜20% compared to PBS, however, despite this loss in signal, changes were detected upon adding serial concentrations of B-IFN-γ. Using bovine plasma samples purified from bovine blood samples, we compared the results from our conducting polymer hydrogel to ELISA method and found correlation between ELISA results and electrochemical signals (FIG. 11D). For real time detection of B-IFN-γ release from bovine blood cells, the antibody immobilized conductive hydrogel was challenged with bovine peripheral blood mononuclear cells (PBMCs). Upon mitogenic stimulation, the change in electrochemical current was observed (FIG. 11E). As controls, the current did not change on the antibody-absent conductive hydrogel with stimulated cells or antibody-immobilized conductive hydrogel with non-stimulated cells. Thus, in the present study, we could confirm our sensor was specific and responsive for B-IFN-γ.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A sensor comprising:
a substrate;
at least one electrode contacting the substrate;
a nanoporous membrane covering the electrode; and
a biorecognition element selected from the group consisting of a peptide, an antibody, an enzyme, and an aptamer, wherein the biorecognition element is covalently bound to the electrode, or covalently bound to a PEDOT random copolymer embedded within the nanoporous membrane, the PEDOT random copolymer having a structure according to Formula I: 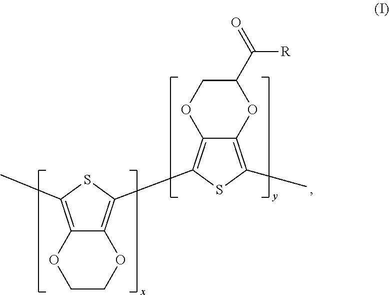
wherein each R is independently selected from the group consisting of —OH and the biorecognition element, wherein at least one R is the biorecognition element, and
x and y are independently an integer of from about 1 to about 1000, wherein the sum of x and y is an integer of from about 2 to about 1000.

2. The sensor of claim 1, wherein the electrode is a micropatterned gold electrode.

3. The sensor of claim 1, wherein the sensor comprises a center working electrode, a surrounding counter electrode, and a reference electrode.

4. The sensor of claim 1, wherein the biorecognition element is an antibody.

5. The sensor of claim 1, wherein the biorecognition element is an aptamer.

6. The sensor of claim 1, wherein the biorecognition element is covalently bound to the PEDOT random copolymer embedded within the nanoporous membrane, wherein the nanoporous membrane comprises a PEG hydrogel having PEG chains with a molecular weight of from about 1000 Da to about 10,000 Da.

7. The sensor of claim 6, wherein the PEG hydrogel comprises PEG chains having a molecular weight of about 6000 Da.

8. The sensor of claim 6, wherein the PEG hydrogel is covalently bound to the substrate.

9. The sensor of claim 6, wherein the ratio of x to y is from about 10:1 to about 1:10.

10. The sensor of claim 1, wherein the nanoporous membrane is an aluminum oxide membrane.

11. The sensor of claim 10, wherein the biorecognition element is covalently linked to the electrode to form a self-assembled monolayer of the biorecognition element on the electrode.

12. The sensor of claim 11, wherein the biorecognition element is an aptamer having a redox reporting moiety and a thiol moiety, wherein the thiol moiety is covalently bound to a gold working electrode.

13. A method for detecting a disease marker in a biological sample comprising contacting a sensor according to claim 1 with the biological sample and detecting the binding of the disease marker to a biorecognition element, thereby detecting the disease marker.

14. The method of claim 13, wherein detecting the binding of the disease marker to the biorecognition element comprises measuring the peak reduction current of the PEDOT random copolymer.

15. The method of claim 13, wherein the binding of the disease marker to the biorecognition element is detected using square wave voltammetry.

16. The method of claim 13, wherein the disease marker is indicative of an infection by tuberculosis or hepatitis C.

17. A conductive hydrogel comprising:
a covalently cross-linked poly(ethylene glycol) (PEG) hydrogel; and
a poly(3,4-ethylenedioxythiophene) (PEDOT) random copolymer embedded within the PEG hydrogel, the PEDOT random copolymer having a structure according to Formula I: 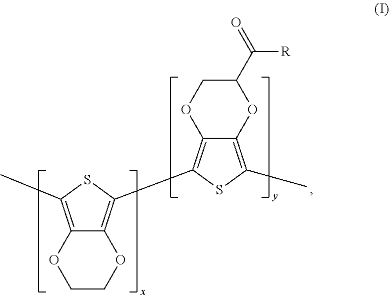
wherein each R is independently selected from the group consisting of —OH and a biorecognition element selected from the group consisting of a peptide, an antibody, an enzyme, and an aptamer, wherein at least one R is the biorecognition element, and
x and y are independently an integer of from about 1 to about 1000, wherein the sum of x and y is an integer of from about 2 to about 1000.

18. The conductive hydrogel of claim 17, wherein the PEG hydrogel comprises PEG chains having molecular weights of from about 1000 Da to about 10,000 Da.

19. The conductive hydrogel of claim 17, wherein the ratio of x to y is from about 10:1 to about 1:10.

20. The conductive hydrogel of claim 19, wherein the covalently cross-linked poly(ethylene glycol) hydrogel is prepared from PEG-diacrylate.
